(19) United States
(12) Patent Application Publication
(10) Pub. No.: US 2026/0317384 A1
(43) Pub. Date: Oct. 15, 2026

(54) SMALL MOLECULE HUMANIN MIMETICS

(71) Applicant: The Regents of the University Of California, Oakland, CA (US)

(72) Inventors: Dongwook Wi, Los Angeles, CA (US); Varghese John, Los Angeles, CA (US); Jesus Campagna, Los Angeles, CA (US); Barbara Jagodzinska, Redwood City, CA (US); Robert Damoiseaux, Los Angeles, CA (US)

(21) Appl. No.: 19/478,977

(22) PCT Filed: Apr. 25, 2024
(86) PCT No.: PCT/US2024/026182

§ 371 Date: Oct. 27, 2025

Related U.S. Application Data
(60) Provisional application No. 63/462,644, filed on Apr. 28, 2023.

(51) Int. Cl. C07K 14/47; A61K 31/517; A61K 38/00; C07D 239/91

(52) U.S. Cl. CPC C07K 14/47; A61K 31/517; C07D 239/91; A61K 38/00

(57) ABSTRACT

The disclosure provides humanin mimetics that increase neuronal p-AKT and enhance p-gp130 as a new therapeutic class for chronic diseases such as Alzheimer's disease.

### CROSS-REFERENCE TO RELATED APPLICATIONS

This application claims the benefit of U.S. Provisional Patent Application No. 63/462,644, filed on Apr. 28, 2023, which is hereby incorporated by reference in its entirety.

### GOVERNMENT SUPPORT

This invention was made with government support under AG068116 awarded by the National Institutes of Health. The government has certain rights in the invention.

### BACKGROUND

Humanin (HN) is a 24-amino acid mitochondrial-derived peptide from the occipital lobe of postmortem sporadic Alzheimer's disease (AD) patient brain tissue that has been shown to protect mitochondria and neurons from amyloid-β (Aβ)-related toxicity. Recently, Yen et al. identified a SNP (rs2854128) by using mitochondrial GWAS in the HN-coding region of the mitochondrial genome and found that in a large sample of older adults, the SNP is associated with accelerated cognitive aging, supporting the concept that HN is an important factor in cognition. A potent HN analog with a substitution of serine-14 to glycine reversed age-related memory impairment in a model of AD. As recently reviewed by Niikura et al., 2022, the 24-residue mitochondrial peptide HN has been found to be protective against neuronal cell death induced by AD-associated insults, such as inhibition of Aβ aggregation and receptor-mediated uptake, tau phosphorylation, and microglia cytokine release. Its protective mechanisms include blocking BAX translocation to mitochondria and regulation of acetylcholine neurotransmission. However, because of the well-known challenges associated with administration of therapeutic peptides, analogs and non-peptidyl molecules that mimic the effects of humanin are needed.

### SUMMARY OF THE INVENTION

In certain aspects, the present disclosure provides compounds represented by formula (I) or a pharmaceutically acceptable salt thereof:

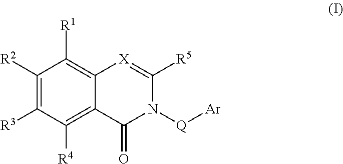

wherein
R¹, R², R³, R⁴, and R⁶ are each independently H, alkyl, alkoxy, halo, nitrile, amino, aminoacyl or aminoalkyl;
R⁵ is H or alkyl;
Q is CH₂ or a bond;
X is CR⁶ or N; and
Ar is phenyl, biphenyl or naphthyl, each optionally substituted; wherein the compound is not

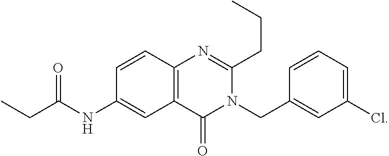 [SMILES: CCCc1nc2ccc(NC(=O)CC)cc2c(=O)n1Cc1cccc(Cl)c1]

In further aspects, the present disclosure provides a truncated humanin peptide consisting of up to 9 amino acid residues.

### BRIEF DESCRIPTION OF THE DRAWINGS

FIG. 1. Functions of HN. HN inhibits Aβ aggregation (1), inhibits receptor-mediated uptake of Aβ (2), activates receptor-mediated anti-cell death signaling cascades (3), blocks Bax translocation to mitochondria (4), attenuates phosphorylation level of tau (5), regulates Ach neurotransmission presynaptically and/or postsynaptically (6), and suppresses cytokine release from microglia.

FIG. 2. Development of p-gp 130 and gp130 AlphaLISA assays (A) Evaluation of p-gp 130 and gp 130 levels in U87-MG and SH-SY5Y cells after treatment with compound 2 by immunoblotting. (B) Principle of AlphaLISA assay. (C) Data of developed gp-130 AlphaLISA assay. Analyte is recombinant gp130 (EC domain). AlphaLISA signal depends on lysate buffer (D) Data of p-pg130 and p-AKT AlphaLISA assays show significant increaste in p-pgp130 and p-AKT signal in U87-MG cells treated with 30 μM of S-propargyl-cyctein (SPRC) for 3 hours. *-p<0.05%, ***-p<0.001 (Student's t-test).

FIG. 3. HTS scattergraph of hits that increase p-gp130 and/or p-Akt>20%.

FIG. 4. Dose response analysis of validated compounds and peptides.

FIG. 5. Schematic for the microfluidic reactor setup.

FIG. 6. Screening of analogs from exploratory medicinal chemistry reveal nanomolar p-gp130 enhancer.

FIG. 7. Layout of 96-well plate for HTS for p-gp130 and p-AKT.

FIG. 8. Reaction scheme for synthesis of the quinazolinone derivatives by flow chemistry synthesis.

FIG. 9. Molecular Docking and Scoring of Protein-Ligand Binding Energy of DDL-814 and DDL-815 (A) Binding of the 9-mer peptide (P3, DDL-814) to the D4-D5 domain of gp130 receptor (pdb: 3L5H) has interactions in the domain and shows a binding AG of −11.77 and a rank score (RS) of −4.29; (B) Binding of the 9mer peptide (Ser→Gly, DDL-815) shows a different mode of binding to the domain region and a weaker binding ΔG-9.72 which is associated with weaker HN mimetic activity through lower p-gp130 and p-Akt.

FIG. 10. Molecular Docking and Scoring of Protein-Ligand Binding Energy of DDL-841 and DDL-842 (A) Binding of dipeptide DDL-841 shows it can bind a similar mode as the 9-mer peptide to gp130 receptor domain and shows a binding RS of −9.07 which could result in the observed increased HN mimetic activity. (B) Binding of tetrapeptide DDL-842 is weaker showing a RS value of −4.18 and is reflected in weak HN mimetic activity.

FIG. 11. Molecular Docking and Scoring of Protein-Ligand Binding Energy of DDL-817 and DDL-834 (A) Binding of small molecule DDL-817 to the gp130 D4-D5 domain similar to the dipeptide DDL-814 and has a AG of −8.09 and a RS of 4.96. (B) Binding of small molecule DDL-834 to the same region shows similar binding with a RS of −5.19.

FIG. 12. Molecular Docking: overlay of small molecule and dipeptide (A) Overlay of small molecule DDL-817 and dipeptide DDL-841 binding to the g130 D4-D5 domain. (B) Overlay of small molecule DDL-834 and dipeptide DDL-841.

### DETAILED DESCRIPTION OF THE INVENTION

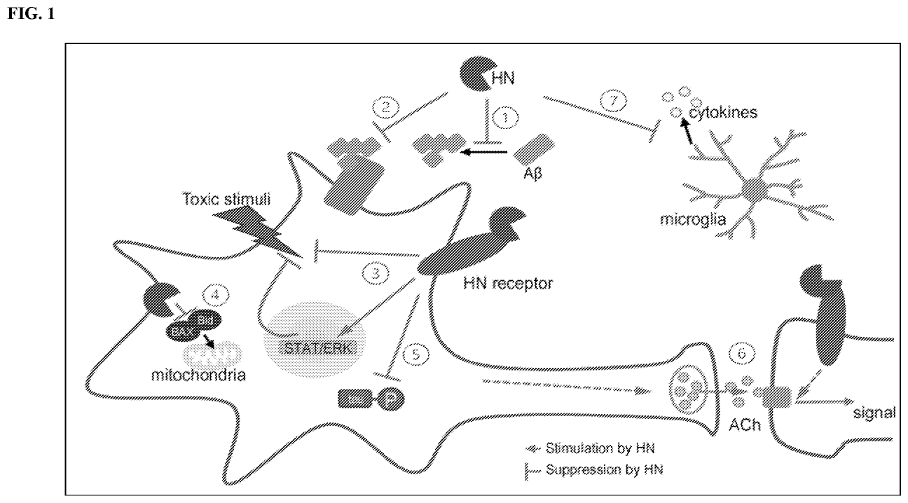

HN exerts its neuroprotective effects through extracellular receptors (FIG. 1). The primary extracellular receptor complex involved in its bioactivity is a trimeric complex involving CNTF, WSX1 and gp130. HN binds to this receptor complex as an agonist and activates the downstream PI3K/Akt pathway, resulting in increased p-Akt levels in neurons. In addition, HN enhancers and HN mimetics are potential therapeutic candidates for enhancement in other chronic diseases such as cardiovascular disease and diabetes.

In certain embodiments, the present disclosure provides compounds represented by formula (I) or a pharmaceutically acceptable salt thereof:

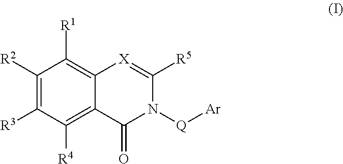

wherein
R¹, R², R³, R⁴, and R⁶ are each independently H, alkyl, alkoxy, halo, nitrile, amino, aminoacyl or aminoalkyl;
R⁵ is H or alkyl;
Q is CH₂ or a bond;
X is CR⁶ or N; and
Ar is phenyl, biphenyl or naphthyl, each optionally substituted; wherein the compound is not

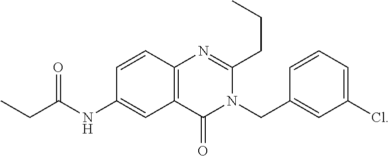 [SMILES: CCCc1nc2ccc(NC(=O)CC)cc2c(=O)n1Cc1cccc(Cl)c1]

In certain embodiments, X is N.

In certain embodiments, R¹ and R⁴ are H.

In certain embodiments, R² is H.

In certain embodiments, R⁵ is methyl.

In certain embodiments, the present disclosure provides compounds represented by formula (II) or a pharmaceutically acceptable salt thereof:

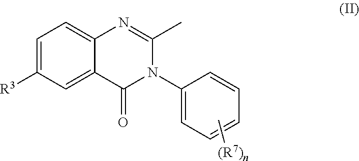

wherein
each R⁷ is independently selected from halo, phenyl, alkyl, acyl, amide, nitro, haloalkyl, nitrile, cycloalkyl, alkoxy, CF₃, ether or alkoxyhalo; and
n is 0, 1, 2, 3, 4, or 5.

In certain embodiments, n is 1. In other embodiments, n is 2.

In certain embodiments, R³ is halo or aminoacyl. In certain such embodiments, R³ is F. In other embodiments, R³ is Br. In yet other embodiments, R³ is C₂H₅(C═O)NH—.

In certain embodiments, R⁷ is Cl. In other embodiments, R⁷ is Br.

In certain embodiments, the compound is selected from:

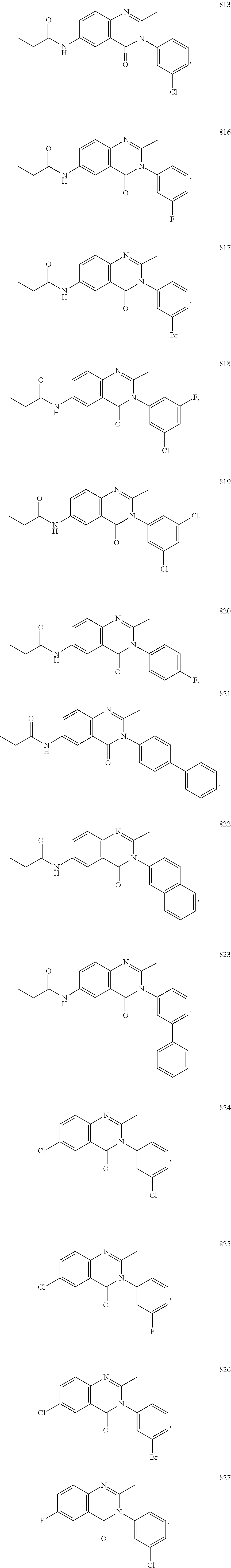

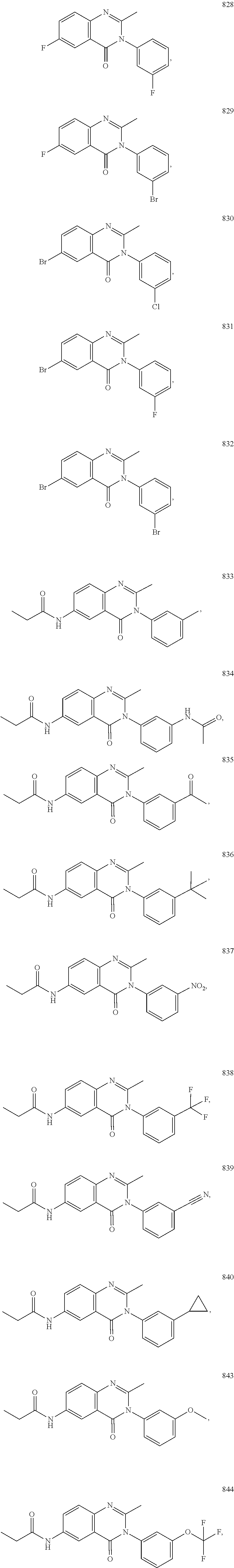

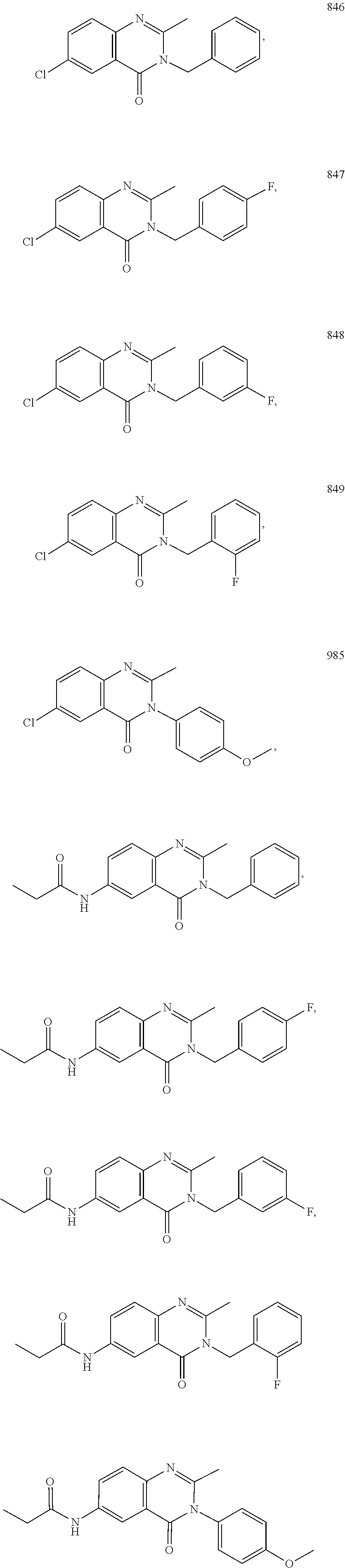

or a pharmaceutically acceptable salt thereof.

In certain embodiments, the present disclosure provides a truncated humanin peptide consisting of up to 9 amino acid residues.

In certain embodiments, the peptide is selected from:
NH₂-Met-Ala-Pro-Arg-Gly-Phe-Ser-Cys-Leu-COOH,
NH₂-Phe-Ser-Cys-Leu-Leu-Leu-Leu-Thr-Ser-COOH,
NH₂-Thr-Ser-Glu-Ile-Asn-Leu-Pro-Val-Lys-COOH (DDL-814),
NH₂-Thr-Gly-Glu-Ile-Asn-Leu-Pro-Val-Lys-COOH (DDL-815),
NH₂-Ile-ASN-Leu-Pro-Val-Lys-Arg-Arg-Ala-COOH,
Z—Thr-Ser-CONH2 (DDL-841),
NH₂-Thr-Gly-Glu-Ile-COOH (DDL-842), and
Phenylacetyl-Thr-Ser-CONH2.

In certain embodiments, the invention provides a pharmaceutical composition comprising a compound of formula I or a truncated humanin peptide consisting of up to 9 amino acid residues and a pharmaceutically acceptable excipient.

In certain embodiments, the invention provides methods of treating or preventing a neurological disease in a subject in need thereof, comprising administering to the subject a compound of formula I or a truncated humanin peptide consisting of up to 9 amino acid residues. In certain such embodiments, the neurological disease is Alzheimer's disease.

In certain embodiments, the invention provides methods of treating or preventing cardiovascular disease in a subject in need thereof, comprising administering to the subject a compound of formula I or a truncated humanin peptide consisting of up to 9 amino acid residues.

In certain embodiments, the invention provides methods of treating or preventing diabetes in a subject in need thereof, comprising administering to a compound of formula I or a truncated humanin peptide consisting of up to 9 amino acid residues.

The ability to readily detect HN peptide levels in the SH-SY5Y cells and in brain tissue of mice was confirmed, whole cell lysates in RIPA buffer were assessed using the Molecular Biosciences ELISA kit MBS7607247.

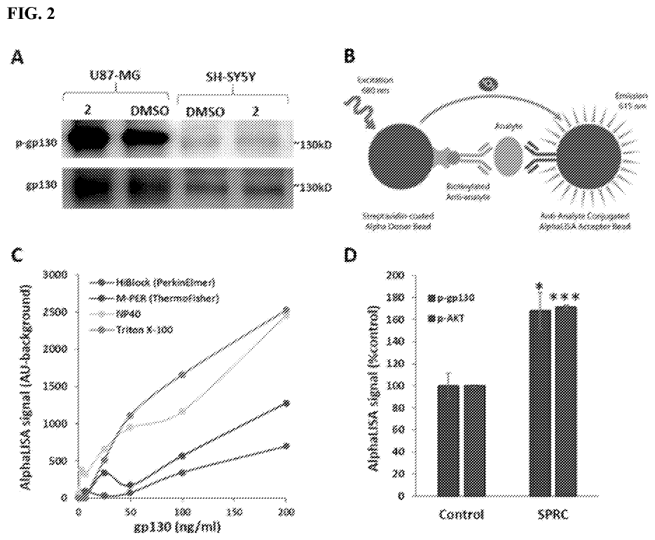

For discovery of HN mimetics, two different cell lines were explored, and it was found that human glioblastoma cells, U87-MG, express more than 10 times more endogenous gp130 compared to SH-SY5Y cells and respond to the treatment with previously identified humanin mimetic, compound 2, by a detectable increase in gp130 phosphorylation at Tyr905 (FIG. 2A). Eight different antibodies (more than 20 combinations) were tested in a course of p-gp130 and gp130 AlphaLISA development. The principle of the AlphaLISA assay is depicted in FIG. 2B. It was found that a pair of antibodies from Sino Biological, Inc., 10974-MM06 (biotinylated) and 10974-RP01 (conjugated to AlphaLISA acceptor beads) represent a valid AlphaLISA pair for total gp-130 detection (FIG. 2C) while another pair of antibodies acquired from MyBioSource, Inc.-MBS2002535 (conjugated to acceptor beads) and MBS8537452 (biotinylated)-enable p-gp130 (Ser 782) detection in U87-MG cell lysates. Lysate buffer composition and treatment conditions have been optimized.

An ELISA assay for the evaluation of the p-gp130 levels was developed. This was a commercial ELISA—DuoSet ELISA from R&D systems (cat #DYC3407-2) and detected the tyrosine phosphorylation of gp130. This ELISA assay was found to be significantly more sensitive and the data produced had low variability. Hence, this assay was used for the HTS screen. In addition, a commercially available p-AKT (1,2,3) AlphaLISA kit was used (PE cat #ALSU-PAKT-B-HV) for the HTS assay.

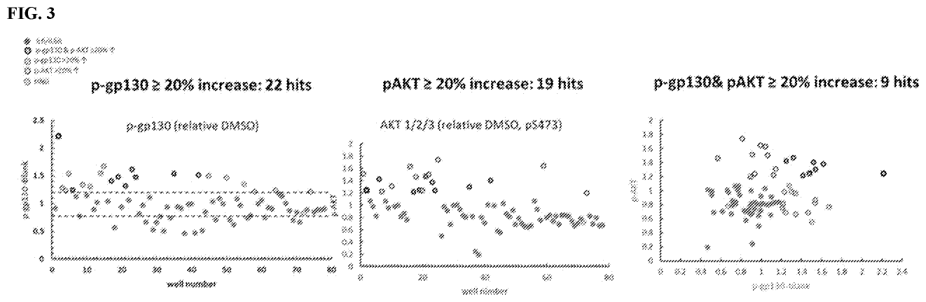

Small molecules that act as HN mimetics represent a new class of AD therapeutics. Through screening, validated hits that increase both p-gp-130 and p-Akt levels were identified. Human glioblastoma cells were used, U87-MG. Several hits that enhance p-gp 130 were identified as well as several compounds that can increase p-AKT (FIG. 3).

Initial HTS found several hits that increased both p-gp130 and p-Akt and some that increased just p-gp130.

Validation testing was conducted of hits that were identified in the HTS screen and only one compound was found that increased both biomarkers (p-gp130 & p-Akt) while four other hits increased one or other of the biomarkers. Further analogues of the validated hit was therefore pursued. A truncated Humanin peptide P3 (see below) was also found that increased p-gp-130.

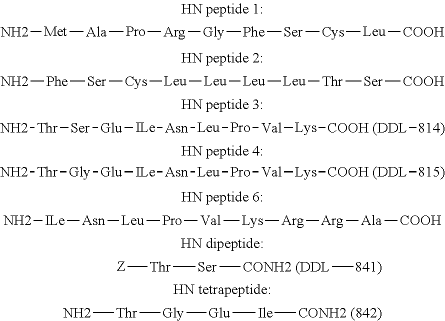

HN derived peptides and peptidomimetics. The peptides in green box increase p-gp130

### Truncated Peptides Based on Humanin (HN) Sequence

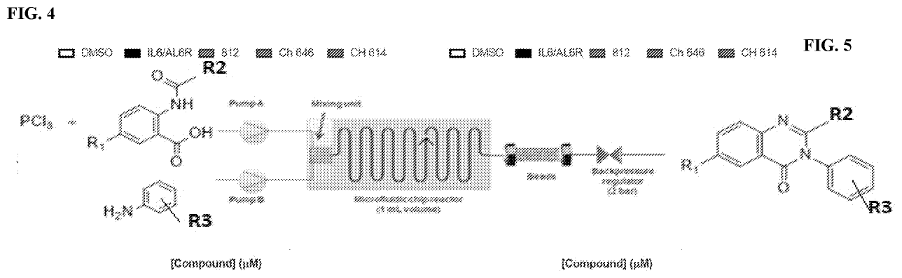

In order to determine if a specific region of the humanin peptide involved in binding and activating the gp130 receptor. Truncated peptides derived from humanin were designed and synthesized (see FIG. 4) and peptide-3 (P3, DDL-814) was shown to activate the gp130 receptor.

Dose Response Testing of Validated Hits for p-Gp130 (p-Tyr)

As shown below dose response testing showed that DDL-813 an analog of Chemistry 16 (DDL-812) is the most potent showing submicromolar activity. In addition, the peptide P3 (DDL-814) also shows good activity. None of the other peptides showed activation of the gp130 receptor.

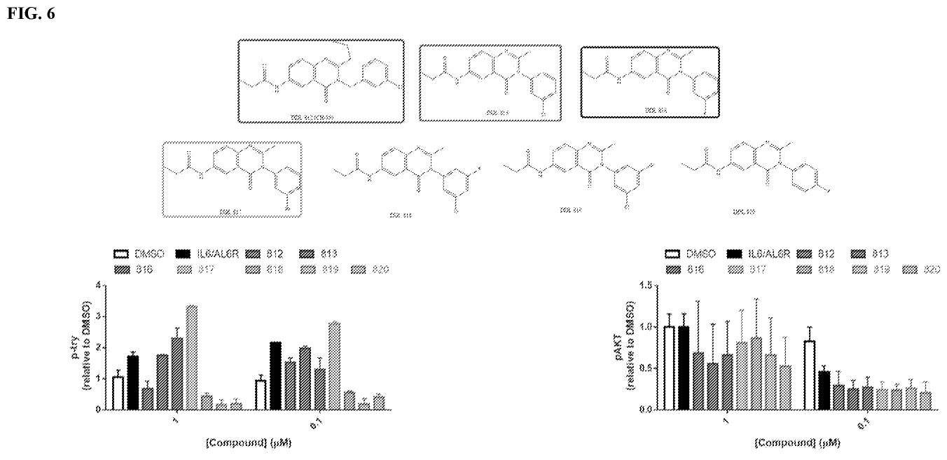

For the analog synthesis, both batch chemistry and flow chemistry (FIG. 5) approaches were used. A distinctive feature is the seamless integration of state-of-the-art flow-chemistry for the structure-activity relationship (SAR) campaign with chemical biology. Analogues of ‘chemistry 16’ were designed and sub micromolar enhancer DDL-817 (EC50<100 nM) found, as shown in the FIG. 6.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro-emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow-release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as a neurological, metabolic, or cardiovascular disease. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂-O-alkyl, —OP(O)(O-alkyl)₂ or —CH₂-OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O) NH—.

The term “acyloxy” is art-recognized and refers to a group represented by the general formula hydrocarbylC(O)O—, preferably alkylC (O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

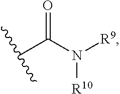

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

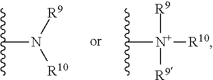

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

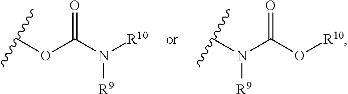

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group-C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a-O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group-OSO₃H, or a pharmaceutically acceptable salt thereof.

The term “sulfonamide” is art-recognized and refers to the group represented by the general formulae.

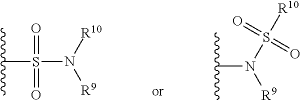

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group-S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group-C(O)SR⁹ or -SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

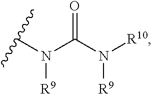

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### Examples

HTS Assay Methodology for p-Gp130 and p-AKT

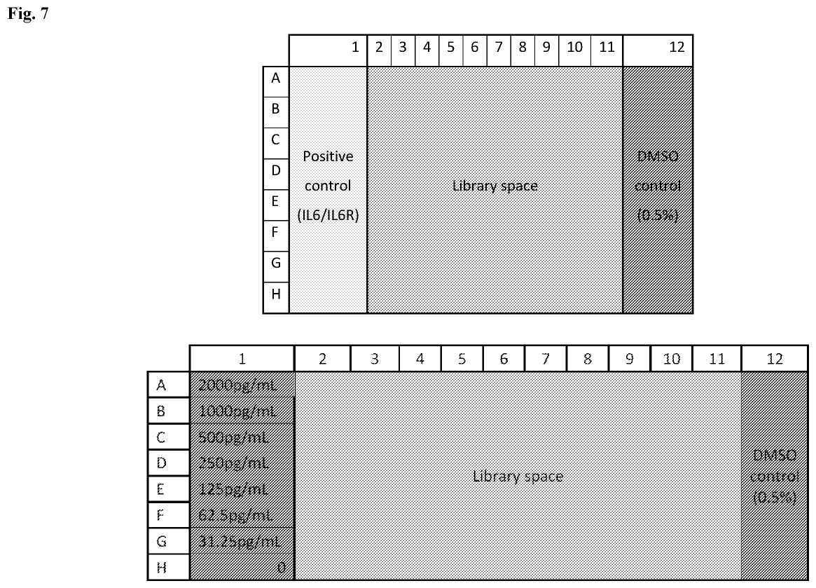

Into a 96 well plate was added 99 μL of warm DMEM/F12 without FBS, then 1 uL of compounds at 1 mM were loaded into the96 well plate. These generated the compounds at 2×. Into a 96 well plate was added IL6/IL6R solution to achieve 6.6×10−4 mg/mL and 3.3×10−4 mg/mL respectively, this is 2× concentration. For this, primary EL6 stock (0.1 mg/mL) was diluted150-fold and IL6R stock (0.1 mg/mL) 300-fold to treat cells with final concentration of 6.6×10−4 mg/mL and 3.3×10−4 mg/mL respectively. 10 ml of Sample Diluent Concentrate 2 (2X)(Part #895891/DYC002)+10 ml ddH₂O+200 ul of 100 mM activated Na₃VO₄ (aliquots at −20C)+20 ul of 10 mg/ml aprotinin (stock at 4C)+20 ul of 10 mg/ml leupeptin (aliquots at −20C)+200 ul PIC-protected from light. Lysis buffer containing 1% NP-40 Alternative, 20 mM Tris (pH 8.0), 137 mM NaCl, 10% glycerol, 2 mM EDTA, 1 mM activated sodium orthovanadate, 10 μg/mL Aprotinin, 10 μg/mL Leupeptin. Placed on ice. Removed the media from the cell plate and add 50 μL of DMEM/F12 without FBS. Then, added 50 μL of the compounds or positive control IL6/IL6R at 2× into the cell plate using the multichannel (FIG. 7). Allowed the treatment to take place for 10 minutes in the incubator at 37° C. and 5% CO₂. Cells were lysed with 130 μL of ice-cold lysis buffer. Placed cells on ice for 15 min, then in the −80° C. overnight. Next, we prepared the ELISA plate by warming coating buffer to RT. 12 strips were coated with capture antibody. For this: 121 μL of 180 μg/mL of capture antibody (R&D DYC3407-2) was added into 21.48 mL of coating buffer. 100 μL of this solution was added to each well, covered with a lead and incubated at RT overnight and then conducted the ELISA assay using the manufacture outlined protocol. The cell lysate from the HTS was prepared using the AlphaLISA buffer to measure levels of p-AKT after compound treatment.

### Molecular Docking Analysis

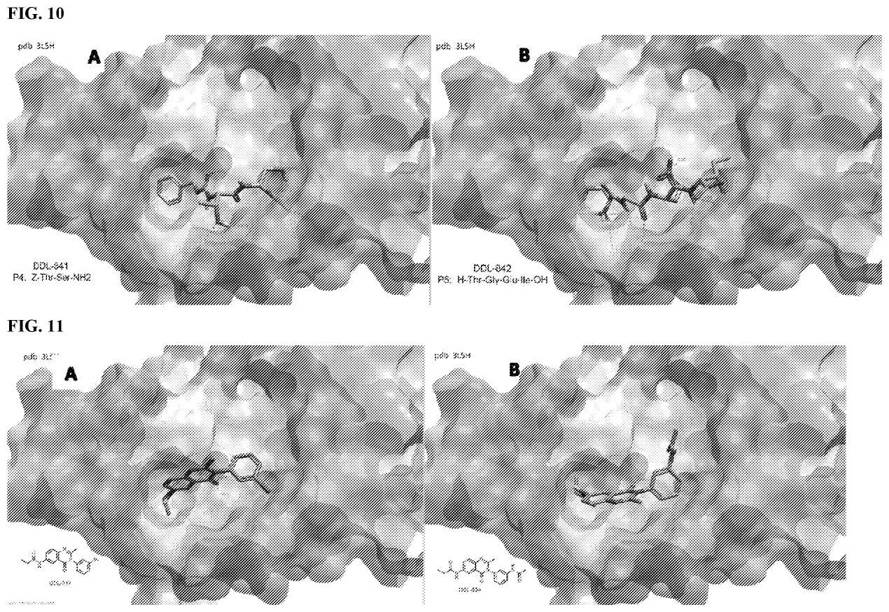

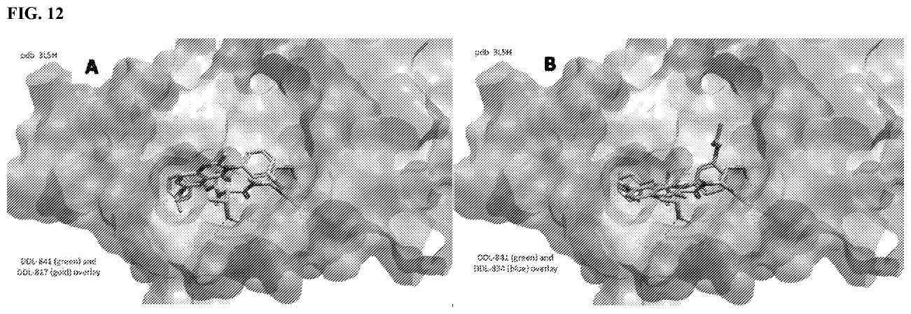

Molecular docking and scoring were performed using Flare (Cresset, v7.2) internal docking algorithm. The docking results quote protein-ligand binding energy (ΔG) and rank-ordering of compounds using Rank Score (RS). The docking was to the D4-D5 domains of the gp130 receptor as shown in FIG. 9-12.

### General Procedure by Batch Chemistry Synthesis Example DDL-813

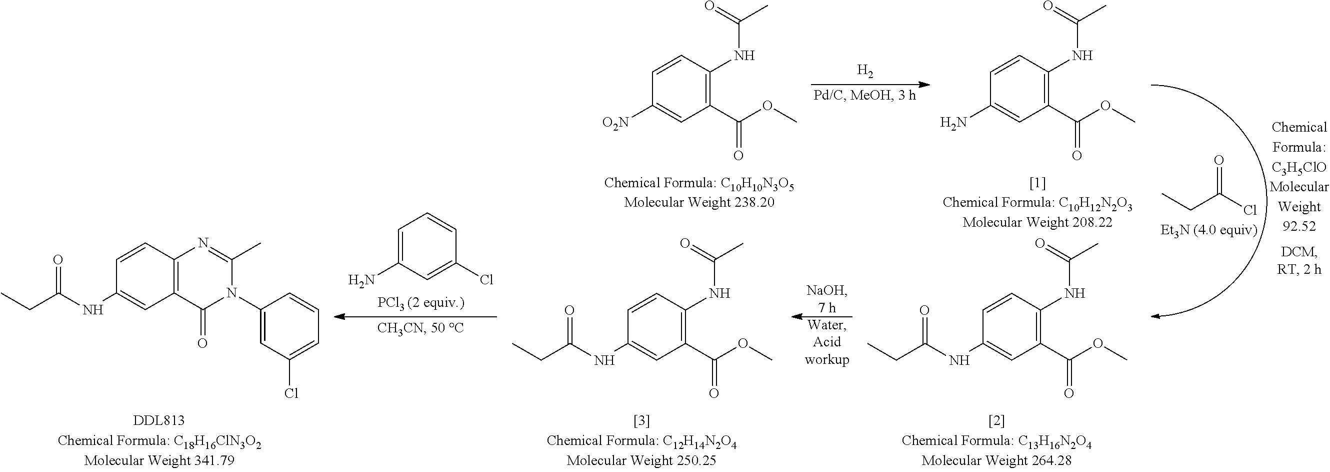

[Intermediate 1]: Methyl-2-amino-5nitrobenzoate (238.2 mg, 1 mmol) was stirred in MeOH (5 mL) under a hydrogen atmosphere with palladium on carbon (10 wt. %) at room temperature and pressure for 3 hours. The mixture was filtered off using a celite plug, with the catalyst being washed with two additional volumes of MeOH before being concentrated in vacuo to yield [1] (157.15 mg of a light-yellow powder, 75.6%). ¹H NMR (400 MHz, DMSO) δ 9.90 (s, 1H), 7.64 (d, J=8.7 Hz, 1H), 7.05 (d, J=2.7 Hz, 1H), 6.72 (dd, J=8.8, 2.8 Hz, 1H), 3.75 (s, 3H), 1.97 (s, 3H). LC-MS m/z [M+H]⁺209.25.

[Intermediate 2]: [1] To a solution of the methyl 2-(acetylamino)-5-aminobenzoate (0.288 mmol) and Triethylamine (1.152 mmol) was added propionyl chloride (0.432 mmol) in DCM (10 mL) under a nitrogen atmosphere while keeping the temperature below 0 degrees. The mixture was warmed to rt and stirred for 2h. Once the reaction was complete as indicated by TLC, 1M HCl (2×, 10 mL) was added, brine (2×, 10 mL), was extracted with DCM, and the organic layer was separated, and concentrated under a vacuum to give the desired product. The resultant crude compound was purified by using a 4 g silica flash column, eluted with DCM: MeOH (time/% MeOH: 0/0, 5/0, 20/10, and 40/100). Purification by flash column chromatography afforded [2]. The fractions that were corresponding to the interest peaks were dried in a speed vacuum to yield (60.2 mg, colorless powder, 79.1%). ¹H NMR (400 MHz, CDCl₃) δ 10.93 (s, 1H), 8.64 (d, J=9.1 Hz, 1H), 8.42 (d, J=2.7 Hz, 1H), 7.47 (dd, J=9.1, 2.7 Hz, 1H), 7.29 (s, 1H), 3.91 (s, 3H), 2.39 (q, J=7.5 Hz, 2H), 2.22 (s, 3H), 1.25 (t, J=7.5 Hz, 3H). LC-MS m/z [M+H]⁺265.17.

[Intermediate 3]: 60.20 mg of [2], 2 mL of THE, 1 mL of H₂O, and 300 μL of 1 N NaOH were added to the vial at 0° C. and the progress of hydrolysis was monitored by thin-layer chromatography (TLC). The emergence of a new spot was monitored by TLC and the gradual disappearance of starting ester. After 7 hr, 320 μL of acid (compared to the initial molar amount of ester) was added dropwise until pH reaches 3. At that moment, the product was extracted using 10 ml of DCM and washed with brine solution two times, dried using anhydrous sodium sulfate, and concentrated under a vacuum to give the desired product (43.68 mg, colorless powder, 76.5%). ¹H NMR (400 MHz, DMSO) δ 10.79 (s, 1H), 9.92 (s, 1H), 8.29 (d, J=9.0 Hz, 1H), 8.23 (d, J=2.6 Hz, 1H), 7.69 (dd, J=9.0, 2.7 Hz, 1H), 2.26 (q, J=7.6 Hz, 2H), 2.06 (s, 3H), 1.03 (t, J=7.6 Hz, 3H). LC-MS m/z [M+H]⁺251.13.

### General Procedure by Batch Chemistry Synthesis DDL-813

A solution of the [3] (0.0816 mmol, 1 Equiv.) in Acetonitrile was added to a solution of the 3-chloro-aniline (0.106 mmol, 1.3 Equiv.) in acetonitrile (2.5 mL) at rt to give a white suspension. PCl3-Phosphorous trichloride (0.163 mmol, 2equiv) was added via a syringe, and the resulting mixture was warmed to 50 degrees and stirred for an additional 2 hr. The mixture was cooled to rt and diluted with DCM. The mixture was quenched by the addition of 1M aqueous HCL solution. The organic layer was separated, washed with 10% Aqueous Sodium Bicarbonate solution, and concentrated under a vacuum to give the DDL 813 without further purification (25.24 mg, colorless powder, 90.5%). ¹H NMR (400 MHz, CDCl₃) δ 8.42 (d, J=8 Hz, 1H), 8.03 (d, J=2 Hz, 2H), 7.67 (d, J=8 Hz, 1H), 7.51-7.16 (m, 4H), 2.26 (q, J=8 Hz, 2H), 2.25 (s, 3H), 1.20 (t, J=8 Hz, 3H). LC-MS m/z [M+H]⁺342.25

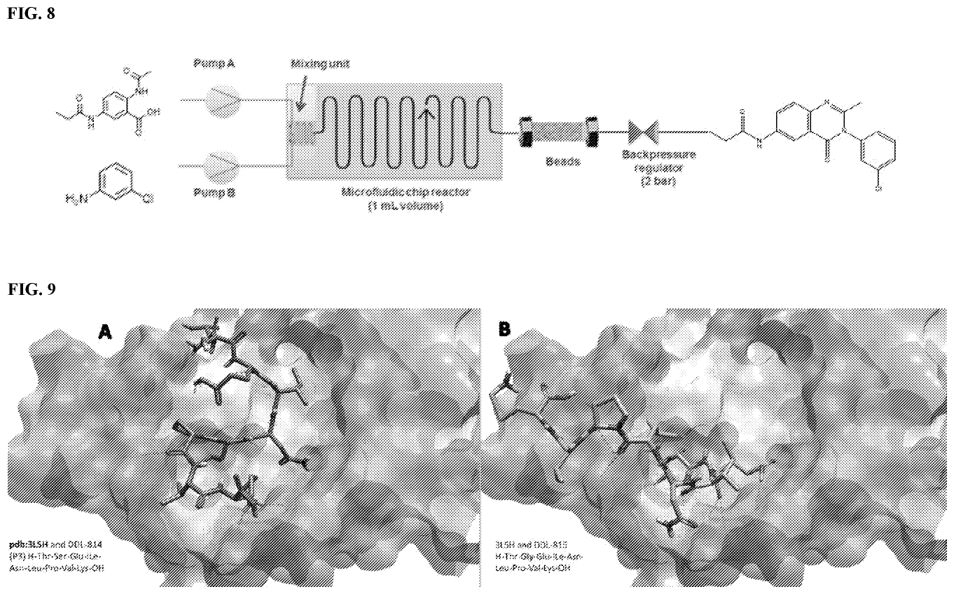

### General Procedure by Flow Chemistry Synthesis DDL-813 (FIG. 8)

Intermediate 3 (1.0 equivalent, 0.05676 mmol) was premixed in 1 mL of THF and PCl₃ (0.5 equivalent, 0.0284 mmol) in a vial 1. 3-chloroaniline (1.1 equivalent, 0.0624 mmol) was premixed in 1 mL of THF in a Vial 2. The solution was pumped through a preheated (60° C. and 2.3 bar pressure) glass microfluidic reactor (Syrris Asia Flow Chemistry Module) at a 250 μL/min flow rate from two pumps to have the desired residence time of one minute in the glass microfluidic reactor. The output of the reactor was passed through a column packed with ion exchange resin (Amberlite IRA-900) to quench the salt produced as a side product and to prevent the clogging of the back-pressure regulator due to salt accumulation otherwise. The output was collected in a flask and concentrated under reduced pressure. The crude reaction mixture was suspended in 15 mL dichloromethane and washed with 3×15 mL sat. NaHCO₃. The organic phase was combined, dried with sodium sulfate, and concentrated under reduced pressure. The obtained crude product was purified using a prepacked silica cartridge on Teledyne CombiFlash Rf 200. Elution with 95:5 DCM-MeOH afforded DDL-813. ¹H NMR (400 MHz, CDCl₃) δ 8.38 (s, 1H), 8.03 (s, 1H), 7.93-7.14 (m, 6H), 2.31 (q, J=8 Hz, 2H), 2.27 (s, 3H), 1.22 (t, J=8 Hz, 3H).

### Synthesis Methods for Analogs by Flow Reaction

DDL-824:6-chloro-3-(3-chlorophenyl)-2-methylquinazolin-4 (3H)-one 4-Chloro-2-acetamidobenzoic acid (1.0 equivalent, 0.117 mmol) was premixed in 2 mL of THF and PCl3 (2 equivalent, 0.234 mmol) in a Vial 1. 3-chloroaniline (1.1 equivalent, 0.1287 mmol) was premixed in 2 mL of THF in a Vial 2. The solution was pumped through a preheated (100° C. and 4 bar pressure) glass microfluidic reactor (Syrris Asia Flow Chemistry Module) at a 100 μL/min total flow rate from two pumps to have the desired residence time of 10 minutes in the glass microfluidic reactor. The output was collected in a flask and concentrated under reduced pressure. The crude was suspended in 5 mL dichloromethane and washed with 3×5 mL sat. NaHCO₃. The organic phase was combined, dried sodium sulfate, and concentrated under reduced pressure. The crude obtained was purified using a prepacked silica cartridge on Teledyne CombiFlash Rf 200. Elution with 95:5 DCM-MeOH afforded 3a (28.4 mg, 80%, white powder). 1H NMR (400 MHz, CDCl₃): δ 8.18 (d, J=4 Hz 1H), 7.69 (s, 1H), 7.52-7.15 (m, 5H), 2.26 (s, 3H). ¹³C NMR (CDCl₃): δ 24.45, 119.16, 126.50, 126.61, 127.64, 128.59, 128.64, 130.03, 131.16,135.84, 138.55, 141.15, 148.30, 155.21, and 161.52. HRMS-ESI (m/z) [M]⁺ calcd for C₁₅H₁₀Cl₂N₂O, 305.02429; found 305.08334. Purity was calculated by dividing the chromatographic peak area for each compound by the sum of all the non-background peak areas in the total ion chromatogram (TIC).

DDL-825:6-chloro-3-(3-fluorophenyl)-2-methylquinazolin-4 (3H)-one 4-Chloro-2-acetamidobenzoic acid (1.0 equivalent, 0.118 mmol) was premixed in 2 mL of THF and PCl₃ (2 equivalent, 0.236 mmol) in a Vial 1. 3-Fluroaniline (1.2 equivalent, 0.142 mmol) was premixed in 2 mL of THF in a Vial 2. The solution was pumped through a preheated (100° C. and 4 bar pressure) glass microfluidic reactor (Syrris Asia Flow Chemistry Module) at a 100 μL/min total flow rate from two pumps to have the desired residence time of 10 minutes in the glass microfluidic reactor. The output was collected in a flask and concentrated under reduced pressure. The crude was suspended in 5 mL dichloromethane and washed with 3×5 mL sat. NaHCO₃. The organic phase was combined, dried with sodium sulfate, and concentrated under reduced pressure. The crude obtained was purified using a prepacked silica cartridge on Teledyne CombiFlash Rf200. Elution with 95:5 DCM-MeOH afforded 825 (25.04 mg, 73.6%, white powder). 1H NMR (400 MHz, CDCl₃): δ 8.18 (d, J-6 Hz, 1H), 7.70 (s, 1H), 7.56-7.06 (m, 5H), 2.28 (s, 3H). ¹³C NMR (CDCl₃): δ 24.44, 117.06, 119.26, 124.12, 126.72, 127.61, 128.67, 131.43, 138.91, 141.12, 148.51,155.17, 161.61, 162.16, and 164.65. 19F NMR (CDCl₃) δ−109.74-−109.80 (m). HRMS-ESI (m/z) [M+H]⁺ calcd for C₁₅H₁₁ClFN₂O, 289.05385; found 289.08334. Purity was calculated by dividing the chromatographic peak area for each compound by the sum of all the non-background peak areas in the total ion chromatogram (TIC).

DDL-826:3-(3-bromophenyl)-6-chloro-2-methylquinazolin-4 (3H)-one 4-Chloro-2-acetamidobenzoic acid (1.0 equivalent, 0.077 mmol) was premixed in 2 mL of THF and PCl3 (2 equivalent, 0.154 mmol) in a Vial 1. 3-Bromoaniline (1.2 equivalent, 0.0924 mmol) was premixed in 2 mL of THF in a Vial 2. The solution was pumped through a preheated (100° C. and 4 bar pressure) glass microfluidic reactor (Syrris Asia Flow Chemistry Module) at a 100 μL/min total flow rate from two pumps to have the desired residence time of 10 minutes in the glass microfluidic reactor. The crude was suspended in 5 mL dichloromethane and washed with 3×5 mL sat. NaHCO₃. The organic phase was combined, dried with sodium sulfate, and concentrated under reduced pressure. The obtained crude product was purified using a prepacked silica cartridge on Teledyne CombiFlash Rf200. Elution with 95:5 DCM-MeOH afforded 826 (11.7 mg, 43%, white powder). 1H NMR (400 MHz, CDCl₃): δ 8.17 (d, J=2 Hz 1H), 7.72-7.22 (m, 6H), 2.29 (s, 3H). ¹³C NMR (CDCl₃): δ 24.50, 119.54, 126.49, 128.57, 128.60, 129.63, 129.84, 130.00,130.34, 131.15, 135.82, 138.58, 148.42, 155.09, and 161.67. HRMS-ESI (m/z) [M]⁺ calcd for C₁₅H₁₉ClBrN₂O, 348.97378; found 349.08334. Purity was calculated by dividing the chromatographic peak area for each compound by the sum of all the non-background peak areas in the total ion chromatogram (TIC).

DDL-827:3-(3-chlorophenyl)-6-fluoro-2-methylquinazolin-4 (3H)-one 4-Fluoro-2-acetamidobenzoic acid (1.0 equivalent, 0.1289 mmol) was premixed in 2 mL of THF and PCl3 (2 equivalent, 0.254 mmol) in a Vial 1. 3-Chloroaniline (1.2 equivalent, 0.155 mmol) was premixed in 2 mL of THF in a Vial 2. The solution was pumped through a preheated (100° C. and 4 bar pressure) glass microfluidic reactor (Syrris Asia Flow Chemistry Module) at a 100 μL/min total flow rate from two pumps to have the desired residence time of 10 minutes in the glass microfluidic reactor. The output was collected in a flask and concentrated under reduced pressure. The crude was suspended in 5 mL dichloromethane and washed with 3×5 mL sat. NaHCO₃. The organic phase was combined, dried with sodium sulfate, and concentrated under reduced pressure. The crude obtained was purified using a prepacked silica cartridge on Teledyne CombiFlash Rf 200. Elution with 95:5 DCM-MeOH afforded 827 (29.5 mg, 79%, white powder). ¹H NMR (400 MHz, CDCl₃): δ 8.26 (d, J=4 Hz, 1H), 7.54-7.15 (m, 6H), 2.27 (s, 3H). ¹³C NMR (CDCl₃): δ 24.51, 112.48, 115.72, 117.45, 126.54, 128.63, 129.95, 131.13, 135.80, 138.65, 149.69,155.12, 161.44, 165.63, and 168.16. ¹⁹F NMR (CDCl₃) δ−102.53-−102.59 (m). HRMS-ESI (m/z) [M+H]⁺ calcd for C₁₅H₁₁ClFN₂O, 289.05385; found 289.16667. Purity was calculated by dividing the chromatographic peak area for each compound by the sum of all the non-background peak areas in the total ion chromatogram (TIC).

DDL-828:6-fluoro-3-(3-fluorophenyl)-2-methylquinazolin-4 (3H)-one 4-Fluoro-2-acetamidobenzoic acid (1.0 equivalent, 0.1 mmol) was premixed in 2 mL of THF and PCl3 (2 equivalent, 0.2 mmol) in a Vial 1. 3-Fluoroaniline (1.2 equivalent, 0.12 mmol) was premixed in 2 mL of THF in a Vial 2. The solution was pumped through a preheated (100° C. and 4 bar pressure) glass microfluidic reactor (Syrris Asia Flow Chemistry Module) at a 100 μL/min total flow rate from two pumps to have the desired residence time of 10 minutes in the glass microfluidic reactor. The output was collected in a flask and concentrated under reduced pressure. The crude was suspended in 5 mL dichloromethane and washed with 3×5 mL sat. NaHCO₃. The organic phase was combined, dried with sodium sulfate, and concentrated under reduced pressure. The obtained crude product was purified using a prepacked silica cartridge on Teledyne CombiFlash Rf 200. Elution with 95:5 DCM-MeOH afforded 828 (14.14 mg, 52%, white powder). ¹H NMR (400 MHz, CDCl₃): δ 8.27 (d, J=2 Hz, 1H), 7.57-7.01 (m, 6H), 2.30 (s, 3H). ¹³C NMR (CDCl₃): δ 24.4, 112.50, 115.63, 115.89, 116.15, 116.92, 124.12, 129.98, 131.45, 138.91, 149.72, 155.19, 161.81, 165.15, and 168.19. ¹⁹F NMR (CDCl₃) δ−102.58-−102.65 (m), 109.80-−102.86 (m), HRMS-ESI (m/z) [M+H]⁺ calcd for C₁₅H₁₁F₂N₂O, 273.08340; found 273.16667. Purity was calculated by dividing the chromatographic peak area for each compound by the sum of all the non-background peak areas in the total ion chromatogram (TIC).

DDL-829:3-(3-bromophenyl)-6-fluoro-2-methylquinazolin-4 (3H)-one 4-Fluoro-2-acetamidobenzoic acid (1.0 equivalent, 0.1 mmol) was premixed in 2 mL of THF and PCl3 (2 equivalent, 0.2 mmol) in a Vial 1. 3-Bromoaniline (1.2 equivalent, 0.12 mmol) was premixed in 2 mL of THF in a Vial 2. The solution was pumped through a preheated (100° C. and 4 bar pressure) glass microfluidic reactor (Syrris Asia Flow Chemistry Module) at a 100 μL/min total flow rate from two pumps to have the desired residence time of 10 minutes in the glass microfluidic reactor. The output was collected in a flask and concentrated under reduced pressure. The crude was suspended in 5 mL dichloromethane and washed with 3×5 mL sat. NaHCO₃. The organic phase was combined, dried with sodium sulfate, and concentrated under reduced pressure.

The crude obtained was purified using a prepacked silica cartridge on Teledyne CombiFlash R/200. Elution with 95:5 DCM-MeOH afforded 829 (24.04 mg, 72%, white powder). ¹H NMR (400 MHz, CDCl₃): ¹H NMR (400 MHz, CDCl₃): δ 8.27 (d, J=2 Hz 1H), 7.68-7.20 (m, 6H), 2.28 (s, 3H). ¹C NMR (CDCl₃): δ 24.56, 112.41, 112.63, 115.64, 115.87, 117.47, 123.54, 127.02, 130.02,129.91, 131.42, 132.88, 138.78, 155.13, and 161.47. 19F NMR (CDCl₃) δ−102.51-−102.57 (m).HRMS-ESI (m/z) [M]⁺ calcd for C₁₅H₁₀BrFN₂O, 333.00333; found 333.16667.

DDL-830:6-bromo-3-(3-chlorophenyl)-2-methylquinazolin-4 (3H)-one 4-Bromo-2-acetamidobenzoic acid (1.0 equivalent, 0.1 mmol) was premixed in 2 mL of THF and PCl3 (2 equivalent, 0.2 mmol) in a Vial 1. 3-Chloroaniline (1.2 equivalent, 0.12 mmol) was premixed in 2 mL of THF in a Vial 2. The solution was pumped through a preheated (100° C. and 4 bar pressure) glass microfluidic reactor (Syrris Asia Flow Chemistry Module) at a 100 μL/min total flow rate from two pumps to have the desired residence time of 10 minute in the glass microfluidic reactor. The output was collected in a flask and concentrated under reduced pressure. The crude was suspended in 5 mL dichloromethane and washed with 3× 5 mL sat. NaHCO₃. The organic phase was combined, dried with sodium sulfate, and concentrated under reduced pressure. The crude obtained was purified using a prepacked silica cartridge on Teledyne CombiFlash Rf200. Elution with 95:5 DCM-MeOH afforded 830 (26.17 mg, 75%, white powder). ¹H NMR (400 MHz, CDCl₃): δ 8.09 (d, q, J-6 Hz, 1H), 7.87 (s, 1H), 7.58 (d, q, J=4 Hz, 1H), 7.52-7.16 (m, 4H), 2.26 (s, 3H). ¹³C NMR (CDCl₃): δ 24.49, 119.55, 126.49, 128.59, 128.60, 129.63, 129.85, 130.00, 130.35, 131.15, 135.82, 138.58, 148.42, 155.09, and 161.67. HRMS-ESI (m/z) [M]⁺ calcd for C₁₅H₁₀BrClN₂O, 348.97378; found 349.00001.

DDL-831:6-bromo-3-(3-fluorophenyl)-2-methylquinazolin-4 (3H)-one 4-Bromo-2-acetamidobenzoic acid (1.0 equivalent, 0.077 mmol) was premixed in 2 mL of THF and PCl₃ (2 equivalent, 0.154 mmol) in a Vial 1. 3-Fluoroaniline (1.2 equivalent, 0.093 mmol) was premixed in 2 mL of THF in a Vial 2. The solution was pumped through a preheated (100° C. and 4 bar pressure) glass microfluidic reactor (Syrris Asia Flow Chemistry Module) at a 100 μL/min total flow rate from two pumps to have the desired residence time of 10 minutes in the glass microfluidic reactor. The output was collected in a flask and concentrated under reduced pressure. The crude was suspended in 5 mL dichloromethane and washed with 3×5 mL sat. NaHCO₃. The organic phase was combined, dried sodium sulfate, and concentrated under reduced pressure. The crude obtained was purified using a prepacked silica cartridge on Teledyne CombiFlash Rf 200. Elution with 95:5 DCM-MeOH afforded 831 (10.8 mg, 42%, white powder). ¹H NMR (400 MHz, CDCl₃): δ 8.11 (d, J=4 Hz 1H), 7.89 (s, 1H), 7.60-7.01 (m, 4H), 2.31 (q, J=8 Hz, 2H), 2.28 (s, 3H). ¹³C NMR (CDCl₃) δ 164.65, 161.95, 155.13, 148.53, 138.85, 131.48, 130.37, 129.91, 129.64, 128.66, 124.10, 119.63, 116.97, 115.99, 24.45. 19F NMR (CDCl₃) δ−109.72-−109.82 (m). HRMS-ESI (m/z) [M+H]⁺ calcd for C₁₅H₁₁BrFN₂O, 333.00333; found 333.16667.

DDL-846:3-benzyl-6-chloro-2-methylquinazolin-4 (3H)-one 4-Chloro-2-acetamidobenzoic acid (1.0 equivalent, 0.117 mmol) was premixed in 2 mL of THF and PCl₃ (2 equivalent, 0.234 mmol) in a Vial 1. Benzylamine (1.2 equivalent, 0.1404 mmol) was premixed in 2 mL of THF in a Vial 2. The solution was pumped through a preheated (120° C. and 4 bar pressure) glass microfluidic reactor (Syrris Asia Flow Chemistry Module) at a 100 μL/min total flow rate from two pumps to have the desired residence time of one minute in the glass microfluidic reactor. The output was collected in a flask and concentrated under reduced pressure. The crude was suspended in 5 mL dichloromethane and washed with 3×5 mL sat. NaHCO₃. The organic phase was combined, dried with sodium sulfate, and concentrated under reduced pressure. The crude obtained was purified using a prepacked silica cartridge on Teledyne CombiFlash Rf200. Elution with 40:60 Hexanes-EtOAc afforded 846 (9.85 mg, 30%, white powder). ¹H NMR (400 MHz, CDCl₃) δ 8.23 (d, J=8.5 Hz, 1H), 7.65 (s, 1H), 7.42 (dd, J=8.6, 2.0 Hz, 1H), 7.35-7.31 (m, 2H), 7.31-7.27 (m, 1H), 7.19 (dd, J=7.1, 1.8 Hz, 2H), 5.38 (s, 2H), 2.55 (s, 3H). ¹³C NMR (CDCl₃) δ 161.97, 1456.15, 148.46, 140.74, 135.72, 129.16, 128.76, 127.97, 127.33, 126.65, 126.52, 118.99, 47.36, 23.64. HRMS-ESI (m/z) [M+H]⁺ calcd for C₁₆H₁₄ClN₂O₁, 285.08334; found 285.07892.

DDL-847:6-chloro-3-(4-fluorobenzyl)-2-methylquinazolin-4 (3H)-one 4-Chloro-2-acetamidobenzoic acid (1.0 equivalent, 0.117 mmol) was premixed in 2 mL of THF and PCl3 (2 equivalent) in a Vial 1. 3-Fluorobenzylamine (1.2 equivalent, 0.225 mmol) was premixed in 2 mL of THF in a Vial 2. The solution was pumped through a preheated (120° C. and 4 bar pressure) glass microfluidic reactor (Syrris Asia Flow Chemistry Module) at a 100 μL/min total flow rate from two pumps to have the desired residence time of one minute in the glass microfluidic reactor. The output was collected in a flask and concentrated under reduced pressure. The crude was suspended in 5 mL dichloromethane and washed with 3×5 mL sat. NaHCO₃. The organic phase was combined, dried with sodium sulfate, and concentrated under reduced pressure. The crude obtained was purified using a prepacked silica cartridge on Teledyne CombiFlash Rf200. Elution with 50:50 Hexanes-EtOAc afforded 847 (12.7 mg, 36%, white powder). ¹H NMR (400 MHz, CDCl₃) δ 8.22 (d, J=8.5 Hz, 1H), 7.64 (s, 1H), 7.42 (dd, J=8.6, 2.0 Hz, 1H), 7.33-7.28 (m, 2H), 7.01-6.94 (m, 2H), 5.36 (s, 2H), 2.54 (s, 3H). ¹³C NMR (CDCl₃) δ 164.55, 161.95, 155.90, 148.23, 140.98, 138.24, 130.83, 128.78, 127.56, 126.51, 122.22, 118.85, 115.08, 113.81, 46.95, 23.51. ¹⁹F NMR (CDCl₃) δ−111.69-−111.75 (m). HRMS-ESI (m/z) [M+H]⁺ calcd for C₁₆H₁₃ClFN₂O₁, 303.25001; found 303.06950.

DDL-848:6-chloro-3-(3-fluorobenzyl)-2-methylquinazolin-4 (3H)-one 4-Chloro-2-acetamidobenzoic acid (1.0 equivalent, 0.117 mmol) was premixed in 2 mL of THF and PCl₃ (2 equivalent, 0.234 mmol) in a Vial 1. 4-Fluorobenzylamine (1.2 equivalent, 0.1404 mmol) was premixed in 2 mL of THE in a Vial 2. The solution was pumped through a preheated (120° C. and 4 bar pressure) glass microfluidic reactor (Syrris Asia Flow Chemistry Module) at a 100 μL/min total flow rate from two pumps to have the desired residence time of one minute in the glass microfluidic reactor. The output was collected in a flask and concentrated under reduced pressure. The crude was suspended in 5 mL dichloromethane and washed with 3×5 mL sat. NaHCO₃. The organic phase was combined, dried with sodium sulfate, and concentrated under reduced pressure. The crude obtained was purified using a prepacked silica cartridge on Teledyne CombiFlash Rf 200. Elution with 55:45 Hexanes-EtOAc afforded 848 (13.5 mg, 38%, white powder). ¹H NMR (400 MHz, CDCl₃) δ 8.22 (d, J=8.5 Hz, 1H), 7.69 (s, 1H), 7.43 (dd, J=8.6, 2.0 Hz, 1H), 7.22-7.17 (m, 2H), 7.06-7.00 (m, 2H), 5.34 (s, 2H), 2.58 (s, 3H). ¹³C NMR (CDCl₃) δ 163.67, 161.95, 161.21, 155.87, 148.40, 140.86, 131.50, 128.72, 128.61, 128.53, 127.44, 126.55,118.95, 116.11, 46.79, 23.61. ¹⁹F NMR (CDCl₃) δ−114.09-−114.16 (m). HRMS-ESI (m/z) [M+H]⁺ calcd for C₁₆H₁₃ClFN₂O₁, 303.25001; found 303.06950.

DDL-849:6-chloro-3-(2-fluorobenzyl)-2-methylquinazolin-4 (3H)-one 4-Chloro-2-acetamidobenzoic acid (1.0 equivalent, 0.117 mmol) was premixed in 2 mL of THF and PCl3 (2 equivalent, 0.389 mmol) in a Vial 1. 2-Fluorobenzylamine (1.2 equivalent, 0.233 mmol) was premixed in 2 mL of THF in a Vial 2. The solution was pumped through a preheated (120° C. and 4 bar pressure) glass microfluidic reactor (Syrris Asia Flow Chemistry Module) at a 100 μL/min total flow rate from two pumps to have the desired residence time of one minute in the glass microfluidic reactor. The output was collected in a flask and concentrated under reduced pressure. The crude was suspended in 5 mL dichloromethane and washed with 3×5 mL sat. NaHCO₃. The organic phase was combined, dried with sodium sulfate, and concentrated under reduced pressure. The crude obtained was purified using a prepacked silica cartridge on Teledyne CombiFlash Rf 200 eluting with 60:40 Hexanes-EtOAc afforded 849 (33%, 11.7 mg, white powder). ¹H NMR (400 MHz, CDCl₃) δ 8.22 (d, J=8.5 Hz, 1H), 7.63 (s, 1H), 7.42 (dd, J=8.6, 2.0 Hz, 1H), 7.31-7.25 (m, 1H), 7.13-7.00 (m, 3H), 5.42 (s, 2H), 2.54 (s, 3H). ¹³C NMR (CDCl₃) δ161.74, 159.08, 156.03, 148.31, 140.90, 129.73, 128.73, 128.12, 127.47, 126.50, 124.92, 122.82,118.88, 115.79, 41.25, 23.24. ¹⁹F NMR (CDCl₃) δ−118.52-−118.58 (m). HRMS-ESI (m/z) [M+H]⁺ calcd for C₁₆H₁₃ClFN₂O₁, 303.16667 found 303.06950.

DDL-985:6-chloro-3-(4-methoxyphenyl)-2-methylquinazolin-4 (3H)-one 4-Chloro-2-acetamidobenzoic acid (1.0 equivalent, 0.303 mmol) was premixed in 5 mL of THF and PCl₃ (2 equivalent, 0.607 mmol) in a Vial 1. 3-Fluoroaniline (1.2 equivalent, 0.364 mmol) was premixed in 2 mL of THF in a Vial 2. The solution was pumped through a preheated (100° C. and 4 bar pressure) glass microfluidic reactor (Syrris Asia Flow Chemistry Module) at a 100 L/min total flow rate from two pumps to have the desired residence time of 10 minutes in the glass microfluidic reactor. The output was collected in a flask and concentrated under reduced pressure. The crude was suspended in 15 mL dichloromethane and washed with 3×15 mL sat. NaHCO₃. The organic phase was combined, dried sodium sulfate, and concentrated under reduced pressure. The crude obtained was purified using a prepacked silica cartridge on Teledyne CombiFlash Rf200. Elution with 95:5 DCM-MeOH afforded 985 (71.9 mg, 79%, brown powder). ¹H NMR (400 MHz, CDCl₃): δ 8.17 (d, J=4 Hz, 1H), 7.65 (s, 1H), 7.40 (d, J-4 Hz, 1H), 7.17-7.03 (m, 4H), 3.87 (s, 3H), 2.24 (s, 3H). 13C NMR (CDCl₃) δ 162.06, 160.20, 156.34, 148.58, 140.78, 130.05, 129.03, 128.69, 127.29, 126.53, 119.40, 116.64, 115.41, 114.93, 55.69, 24.60. HRMS-ESI (m/z) [M+H]⁺ calcd for C₁₆H₁₃ClN₂O₂, 301.07383; found 301.33334.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound represented by formula (I) or a pharmaceutically acceptable salt thereof: 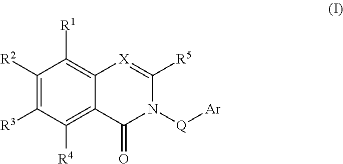
wherein
R¹, R², R³, R⁴, and R⁶ are each independently H, alkyl, alkoxy, halo, nitrile, amino, aminoacyl or aminoalkyl;
R⁵ is H or alkyl;
Q is CH₂ or a bond;
X is CR⁶ or N; and
Ar is phenyl, biphenyl or naphthyl, each optionally substituted; wherein the compound is not

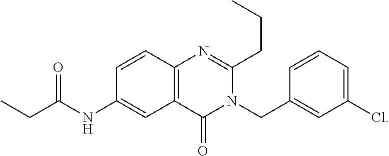 [SMILES: CCCc1nc2ccc(NC(=O)CC)cc2c(=O)n1Cc1cccc(Cl)c1]

2. The compound of claim 1, wherein X is N.

3. The compound of claim 1 or 2, wherein R¹ and R⁴ are H.

4. The compound of any one of claims 1-3, wherein R² is H.

5. The compound of any one of claims 1-4, wherein R⁵ is methyl.

6. The compound of any one of claims 1-5, wherein the compound is represented by formula (II): 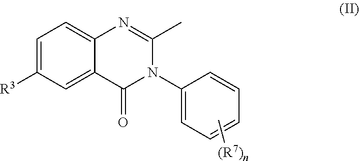
wherein
each R⁷ is independently selected from halo, phenyl, alkyl, acyl, amide, nitro, haloalkyl, nitrile, cycloalkyl, alkoxy, CF₃, ether or alkoxyhalo; and
n is 0, 1, 2, 3, 4, or 5.

7. The compound of claim 6, wherein n is 1.

8. The compound of claim 6, wherein n is 2.

9. The compound of any one of claims 1-8, wherein R³ is halo or aminoacyl.

10. The compound of any one of claims 1-9, wherein R³ is F.

11. The compound of any one of claims 1-10, wherein R³ is Br.

12. The compound of any one of claims 1-11, wherein R³ is C₂H₅ (C═O) NH—.

13. The compound of any one of claims 1-12, wherein R⁷ is Cl.

14. The compound of any one of claims 1-13, wherein R⁷ is Br.

15. A compound selected from: 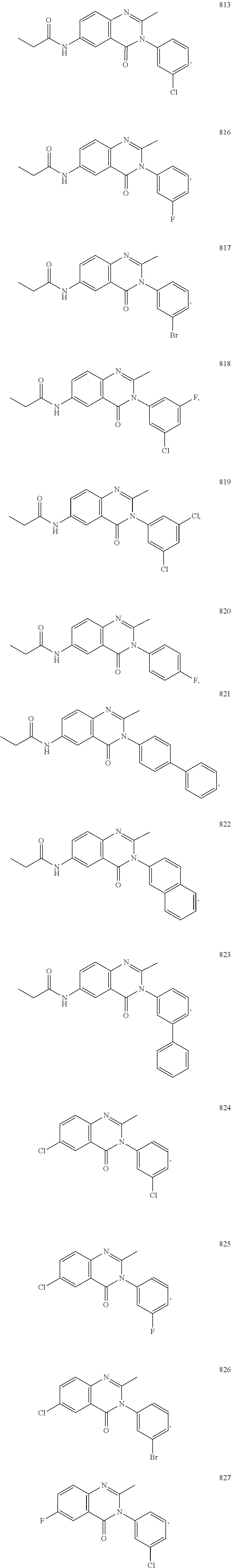 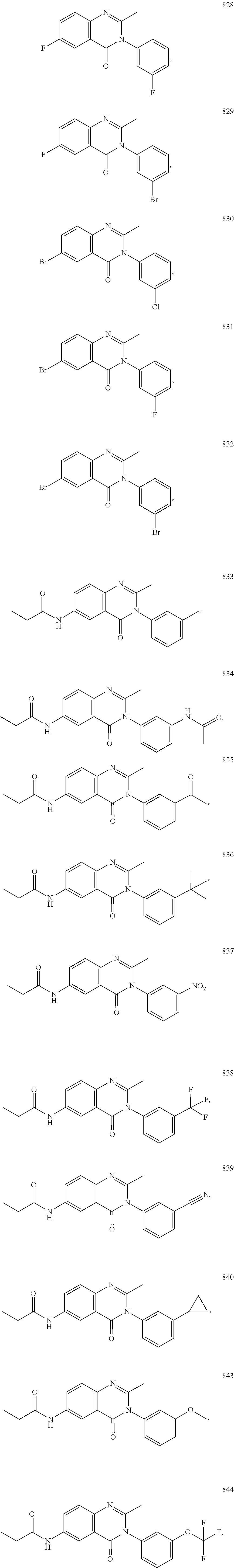 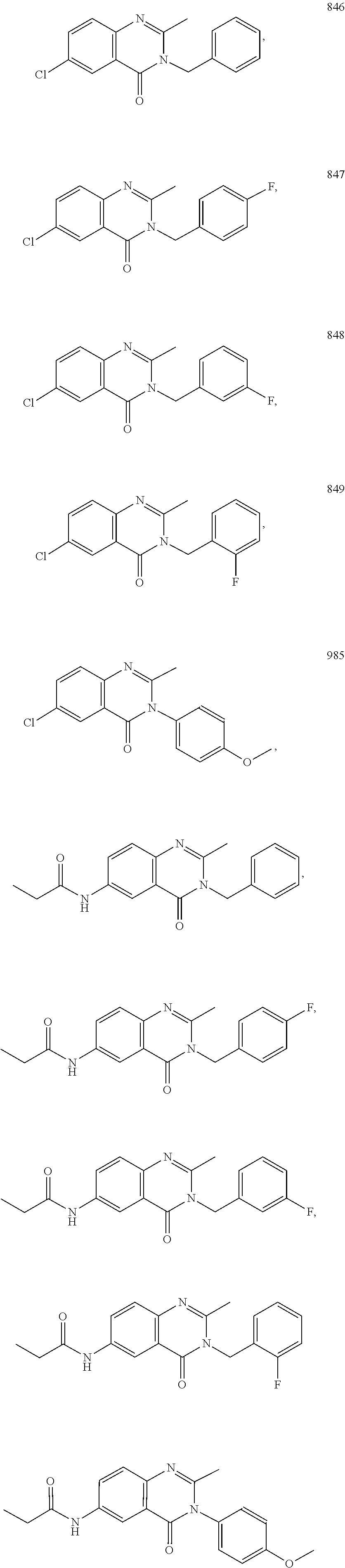
or a pharmaceutically acceptable salt thereof.

16. A truncated humanin peptide consisting of up to 9 amino acid residues.

17. A peptide of claim 16 selected from:
NH₂-Met-Ala-Pro-Arg-Gly-Phe-Ser-Cys-Leu-COOH,
NH₂-Phe-Ser-Cys-Leu-Leu-Leu-Leu-Thr-Ser-COOH,
NH₂-Thr-Ser-Glu-Ile-Asn-Leu-Pro-Val-Lys-COOH (DDL-814),
NH₂-Thr-Gly-Glu-Ile-Asn-Leu-Pro-Val-Lys-COOH (DDL-815),
NH₂-Ile-ASN-Leu-Pro-Val-Lys-Arg-Arg-Ala-COOH,
Z—Thr-Ser-CONH2 (DDL-841),
NH₂-Thr-Gly-Glu-Ile-COOH (DDL-842), and
Phenylacetyl-Thr-Ser-CONH2.

18. A pharmaceutical composition comprising a compound of any one of claims 1-17 and a pharmaceutically acceptable excipient.

19. A method of treating or preventing a neurological disease in a subject in need thereof, comprising administering to the subject a compound of any one of claims 1-17.

20. The method of claim 19, wherein the neurological disease is Alzheimer's disease.

21. A method of treating or preventing cardiovascular disease in a subject in need thereof, comprising administering to the subject a compound of any one of claims 1-19.

22. A method of treating or preventing diabetes in a subject in need thereof, comprising administering to the subject a compound of any one of claims 1-19.
